(19) United States
(12) Patent Application Publication
(10) Pub. No.: US 2014/0277450 A1
(43) Pub. Date: Sep. 18, 2014

(54) APPARATUS AND METHODS FOR AOL AND DRL RECONSTRUCTION OF CMC JOINTS

(71) Applicant: Mark J. Warburton, High Point, NC (US)

(72) Inventor: Mark J. Warburton, High Point, NC (US)

(21) Appl. No.: 14/213,348

(22) Filed: Mar. 14, 2014

Related U.S. Application Data
(60) Provisional application No. 61/783,259, filed on Mar. 14, 2013.

(51) Int. Cl. A61B 17/17; A61F 2/08

(52) U.S. Cl. CPC A61B 17/1739; A61F 2/08
USPC 623/13.14; 606/96; 623/13.11

(57) ABSTRACT

A surgical pin guide for reconstruction of a carpo-metacarpal (CMC) joint of a hand includes: a guide body having first and second opposed second end portions; a first pin receiving member connected to the first end portion of the guide body, the first pin receiving member having an elongated channel extending therethrough; and a second pin receiving member connected to the second end portion of the guide body, the second pin receiving member having an elongated channel extending therethrough. The first pin receiving member channel is sized and configured to receive a proximal end portion of a first pin that has a distal end portion residing in a trapezium of a hand. The second pin receiving member channel is sized and configured to receive and align a second pin for insertion into a first metacarpal of the hand.

### RELATED APPLICATIONS

This application claims priority from U.S. Provisional Application No. 61/783,259, filed Mar. 14, 2013, the disclosure of which is hereby incorporated herein in its entirety.

### BACKGROUND

Thumb prehension which is used for grasp sets the human apart from lower mammals. The joint of the thumb which allows for rotation (opposition) is the carpo-metacarpal (CMC) joint of the hand. The trapezium is a carpal bone but, embryologically, is a metacarpal, which articulates with the 1st metacarpal (MC) of the thumb. The CMC has a double saddle joint, allowing almost 120 degrees of rotation. This mobility is advantageous in some respects, but in a certain subset of patients, can lead to early arthritis. Degenerative arthritis of the CMC joint of the thumb is a common finding in patients with thumb pain. The disease process has a predictable course starting with instability and synovitis (inflammation of the soft tissue lining of the joint) and progressing to degenerative arthritis over the course of many years. The main stabilizers of the CMC joint are the anterior oblique ligament (AOL) and the dorsoradial ligament (DRL). Attenuation or laxity of these ligaments results in dorso-radial subluxation of the CMC joint. This causes incongruity of the joint and increased wear of the chondral surface of the trapezium mainly, and to a lesser extent the base of the first metacarpal. Degenerative arthritis results from this wear and is graded according to the Eaton-Littler classification. If the arthritis is minimal (Eaton-Littler stage I or II), reconstruction (augmentation) of the AOL and DRL can improve the symptoms of pain and slow the progression of arthritis.

### SUMMARY

Some embodiments of the invention are directed to a surgical pin guide for reconstruction of a carpo-metacarpal (CMC) joint of a hand. The pin guide includes: a guide body having first and second opposed second end portions; a first pin receiving member connected to the first end portion of the guide body, the first pin receiving member having an elongated channel extending therethrough; and a second pin receiving member connected to the second end portion of the guide body, the second pin receiving member having an elongated channel extending therethrough; wherein the first pin receiving member channel is sized and configured to receive a proximal end portion of a first pin that has a distal end portion residing in a trapezium of a hand, and wherein the second pin receiving member channel is sized and configured to receive and align a second pin for insertion into a first metacarpal of the hand.

The pin guide may include a removable sleeve within the second pin receiving member channel, the removable sleeve having an elongated channel extending therethrough, wherein the removable sleeve channel is sized and configured to receive the second pin, and wherein, when the removable sleeve is removed, the second pin receiving member channel is sized configured to receive and align a third pin for insertion into the first metacarpal of the hand. The third pin may have a diameter that is greater than a diameter of the second pin. In some embodiments, the first pin receiving member channel has a diameter of between about 1 mm and 1.55 mm; the removable sleeve member channel has a diameter of between about 1 mm and 1.55 mm; and the second pin receiving member channel has a diameter of between about 2.2 mm and 2.55 mm.

In some embodiments, the first pin receiving member is fixedly connected to the guide body. In some embodiments, the second pin receiving member is adjustably connected to the guide body. The second pin receiving member may be slidable and/or translatable along a length direction of the guide body away from and toward the first pin receiving member. The guide body may include an elongated slot that receives a projection of the second pin receiving member, the projection being slidable within the slot. In some embodiments, the first pin receiving member channel defines a first axis, the second pin receiving member channel defines a second axis, and the second pin receiving member is pivotable so as to adjust an angle between the first axis and the second axis.

The pin guide may include a locking mechanism for locking and/or securing the second pin receiving member in place on the guide body. The guide body may have first and second opposing faces, the second pin receiving member residing adjacent the first face and the locking mechanism residing against or adjacent the second face. The first pin receiving member may be fixedly disposed on or integrally formed with the first face.

Other embodiments of the invention are directed to a method for performing anterior oblique ligament (AOL) and dorsoradial ligament (DRL) reconstruction of the carpo-metacarpal (CMC) joint of a patient, the method comprising: advancing a distal end portion of a first guide pin in a trapezium of the patient; positioning a guide having first and second pin receiving members such that a proximal end of the first guide pin is received in the first pin receiving member; inserting a distal end portion of a second guide pin in the second pin receiving member of the guide; and advancing the distal end portion of the second guide pin through the second pin receiving member of the guide and into a first metacarpal of the patient.

In some embodiments, the method includes: retracting the distal end portion of the second guide pin from the first metacarpal of the patient and from the second pin receiving member of the guide; inserting a distal end portion of a third guide pin in the second pin receiving member of the guide; and advancing the distal end portion of the third guide pin through the second pin receiving member of the guide and into the first metacarpal of the patient. A diameter of the third guide pin may be greater than a diameter of the second guide pin. The guide may include a removable sleeve in the second pin receiving member, and the method may include removing the removable sleeve from the second pin receiving member prior to inserting a distal end portion of a third guide pin in the second pin receiving member of the guide. The method may include removing the guide after advancing the distal end portion of the third guide pin through the second pin receiving member of the guide and into the first metacarpal of the patient.

In some embodiments, the method includes: drilling a channel or hole in the trapezium using the first guide pin; and drilling a channel or hole in the first metacarpal using the third guide pin. The method may include removing the first and third guide pins after drilling the holes in the trapezium and the first metacarpal.

In some embodiments, the method includes passing a graft through the hole drilled in the trapezium and the hole drilled in the first metacarpal. This may be done by performing a highly technical blind digital retrieval of a nitinol wire attached to a tendon graft. The graft may be a tendon graft or a palmaris longus graft. The method may include: anchoring the graft in the hole drilled in the trapezium using a first screw; and anchoring the graft in the hole drilled in the first metacarpal using a second screw. The graft may be passed through the hole drilled in the trapezium and the hole drilled in the first metacarpal such that the first screw anchors a length of the graft in the hole drilled in the trapezium and such that the second screw anchors first and second opposing ends of the graft in the hole drilled in the first metacarpal.

In some embodiments, the method includes adjusting the second pin receiving member of the guide prior to and/or during the step of inserting a distal end portion of a second guide pin in the second pin receiving member of the guide and/or the step of advancing the distal end portion of the second guide pin through the second pin receiving member of the guide and into a first metacarpal of the patient. In some embodiments, the guide comprises a guide body with the second pin receiving member adjustably connected to the guide body, and wherein adjusting the second pin receiving member comprises translating the second pin receiving member along the guide body and/or pivoting the second pin receiving member relative to the guide body.

Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE FIGURES

FIG. 1 is a side view of a pin guide according to some embodiments.

FIG. 2 is an enlarged top view of the pin guide of FIG. 1.

FIG. 3 is an x-ray showing drill guide pins received in the pin guide of FIG. 1 and advanced in the trapezium and first metacarpal of a patient according to some embodiments.

FIG. 4 is a model illustrating a graft passing through and anchored in holes drilled using the drill guide pins of FIG. 3 according to some embodiments.

FIG. 5 is an alternative view of the model of FIG. 4.

FIGS. 6-18 illustrate a sequence of steps that can be used for a surgical procedure according to some embodiments.

### DETAILED DESCRIPTION OF EMBODIMENTS OF THE INVENTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the expression “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is noted that any one or more aspects or features described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

As used herein, the term “about” used in connection with a claimed value means +/−10% or +/−20% of the claimed value in various embodiments.

A pin guide 10 according to some embodiments is illustrated in FIGS. 1-3. The pin guide 10 includes a guide body 12. A first pin receiving member 14 is connected to a first end portion 16 of the guide body 12. A second pin receiving member 18 is connected to a second end portion 20 of the guide body 12. The first pin receiving member 14 includes an elongated channel 14c extending therethrough and the second pin receiving member 18 includes an elongated channel 18c extending therethrough (FIG. 2). As will be described in greater detail below, the pin guide 10 may be used during CMC joint augmentation or reconstruction surgery. In some embodiments, a first pin that has been inserted into a trapezium of a patient may be received in the first pin receiving member channel 14c. Second and third pins may be received in the second pin receiving member channel 18c, with the second pin receiving member 18 configured to align the second and third pins for insertion into a first metacarpal of the patient.

A removable cylindrical sleeve 24 may be snugly and/or slidably received in the second pin receiving member channel 18c. The removable sleeve 24 has a channel 24c extending therethrough (FIG. 1). The removable sleeve channel 24c has a diameter that is smaller than a diameter of the second pin receiving member channel 18c. In this regard, the second pin may be received in the removable sleeve channel 24c and aligned for insertion into the first metacarpal. Once the second pin has been inserted at a proper location/position, the second pin and the removable sleeve 24 may be removed from the second pin receiving member channel 18c. The third pin having a diameter greater than a diameter of the second pin may then be received in the second pin receiving member channel 18c for insertion into the first metacarpal.

This is exemplified in FIG. 3. A distal end portion 22d of a first guide pin 22 is advanced or inserted into a trapezium T of a patient. Fluoroscopy or other suitable technique may be used to facilitate advancement/insertion of the first pin 22 to the proper location/position in the trapezium T. The guide 10 is provided and a proximal end 22p portion of the first pin 22 may be received in the first pin receiving member 14 (i.e., in the channel 14c). A distal end portion 26d of a second pin 26 may be received in the removable sleeve 24 (i.e., the channel 24c) with the removable sleeve positioned in the second pin receiving member channel 18c (FIG. 1). With the second pin receiving member 18 in the proper orientation and/or position, the second pin receiving member 18 is configured to align the second pin 26 for advancement or insertion into the first metacarpal MC of the patient. Fluoroscopy or other suitable technique may be used to facilitate advancement/insertion of the distal end 26d of the second pin 26 to the proper location/position in the first metacarpal MC. As will be described below, the second pin receiving member 18 of the guide 10 may be adjustably held on the guide body 12. In this regard, the second pin receiving member 18 may be adjusted before and/or during advancement of the second pin 26 into the first metacarpal MC. Once the position of the second pin 26 in the first metacarpal MC is deemed satisfactory (e.g., via fluoroscopy), the second pin 26 and the removable sleeve 14 may be removed from the second pin receiving member 18 of the guide 10. A distal end portion 28d of a third pin 28 may be received in the second pin receiving member 18 (i.e., the channel 18c) and advanced or inserted to extend through the channel 18c and to the proper position in the first metacarpal MC.

In some embodiments, the first pin 22 has a diameter of between 1 mm and 1.05 mm, typically 1.04 mm or about 1.04 mm. In some embodiments, the second pin 26 has a diameter of between 1 mm and 1.05 mm, typically 1.04 mm or about 1.04 mm. In some embodiments, the third pin 28 has a diameter that is at least two times greater than the diameter of the first pin 22 and/or the second pin 26. In some embodiments, the third pin 28 has a diameter of 2.4 mm or about 2.4 mm. In some embodiments, the first pin has a diameter of between 1 mm and 1.55 mm. In some embodiments, the second pin has a diameter of between 1 mm and 1.55 mm. In some embodiments, the third pin has a diameter of between 2.2 mm and 2.55 mm.

The first pin receiving member channel 14c may have a diameter that is about the same or slightly greater than that of the first pin 22. The removable sleeve channel 24c may have a diameter that is about the same or slightly greater than that of the second pin 26. The removable sleeve 24 may have an outer diameter that is about the same or slightly less than the diameter of the second pin receiving member channel 18c. The second pin receiving member channel 18c may have a diameter that is about the same or slightly greater than that of the third pin 28. In various embodiments, the first pin receiving member channel 14c and/or the removable sleeve channel 24c have a diameter of between about 1.05 mm and 1.50 mm, between about 1.05 mm and 1.40 mm, between about 1.05 mm and 1.26 mm, between about 1.01 mm and 2 mm, between about 1.01 mm and 1.90 mm, and between about 1.01 mm and 1.75 mm. In various embodiments, the second pin receiving member channel 18c has a diameter of between about 2.41 mm and 2.90 mm, between about 2.41 mm and 2.75 mm, between about 2.41 mm and 2.66 mm, between about 2.21 mm and 3 mm, between about 2.21 mm and 2.90 mm, and between about 2.21 mm and 2.80 mm.

Any suitable instrument known to those of skill in the art may be used to advance the first, second and third pins 22, 26, 28; an exemplary suitable instrument is an powered drill.

The pins 22, 26, 28 may be used as drill guide pins. That is, after the pins 22, 26, 28 have been advanced to their proper position, one or more of the pins may be used to accept and guide drills for making an appropriate sized channel or hole in the trapezium and first metacarpal. For example, if the first pin 22 has a diameter of 1.04 mm or about 1.04 mm, a hole having a diameter of 3 mm or about 3 mm may be drilled in the trapezium. If the third pin 28 has a diameter of 2.4 mm or about 2.4 mm, a hole having a diameter of 4 mm or about 4 mm may be drilled in the first metacarpal. In some embodiments, a channel or hole having a diameter of between about 3 and 4 mm may be drilled in the trapezium. In some embodiments, a channel or hole having a diameter of between about 4 and 4.5 mm may be drilled in the first metacarpal. The holes can be used to accept screws, suture anchors or other fasteners for anchoring a graft, as will be described below.

As noted above, the second pin receiving member 18 may be adjustable on the guide body 12. The second pin receiving member 18 may be configured to translate along the guide body 12 as shown by the arrow T in FIG. 1 (i.e., move away from and toward the first pin receiving member 14). The second pin receiving member 18 may also be pivotable as shown by the arrow P in FIG. 1. As shown in FIG. 1, the first pin receiving member channel 14c may define a first axis A1 and the second pin receiving member channel 18c may define a second axis A2. Pivoting the second pin receiving member 18 in the direction P and/or translating the second pin receiving member in the direction T may alter the angle between the axes A1, A2, and as a result the angle between pins received in the channels 14c, 16c, 24c. In some embodiments, the second pin receiving member 18 is adjustable such that the axes Al and A2 are parallel to slightly converging. It will be appreciated that, due to the adjustable nature of the second pin receiving member 18, the guide 10 may be adjustable for the variation in anatomy size for different patients.

Referring to FIG. 2, the guide body 12 may have an end-to-end length L of between about 1 and about 3 inches. In some embodiments, the length L is less than about 2.5 inches and, in some embodiments, between 1 and 2 inches, such as about 1 inch, 1.25 inches, 1.5 inches, 1.75 inches and 2 inches. In some embodiments, and as illustrated in FIG. 1, the guide body 12 has an arcuate profile or shape when viewed from the side.

In the embodiment illustrated in FIG. 1, the guide body 12 includes a slot 30. The second pin receiving member 18 may include a projection 18p (FIG. 2) that is configured to translate within the slot 30 such that the second pin receiving member 18 is adjustable in the direction T.

In some embodiments, a center or pivot point C of the second pin receiving member 18 is slidable or translatable a distance of between about 0.25 inches and 1 inch along the direction T. In some embodiments, the center or pivot point C of the second pin receiving member 18 is slidable or translatable a distance of between about 0.25 and 0.75 inches, and in some embodiments, about 0.5 inches, in the direction T. In some embodiments, the second pin receiving member 18 may be translated in the slot 30 toward the first pin receiving member 14 such that the center or pivot point C of the second pin receiving member 18 is between 0.25 and 0.75, and in some embodiments about 0.5 inches, from the first pin receiving member 14. In some embodiments, the second pin receiving member 18 may be translated in the slot 30 away from the first pin receiving member 14 such that the center or pivot point C of the second pin receiving member 18 is between about 0.75 and about 1.25, and in some embodiments, about 1 inch, from the first pin receiving member 14.

Referring to FIG. 2, the guide 10 can include a locking mechanism 32 configured to the lock or secure the second pin receiving member 18 in place (for example, after adjustment). In the illustrated embodiment, the projection 18p is threaded and the locking mechanism 32 is a nut threadingly engaged with the projection 18p, although any suitable locking mechanism known to those skilled in the art may be employed. The locking mechanism 32 may be manipulated (e.g., loosened) to allow an operator to translate the second pin receiving member 18 in the direction T and/or pivot the second pin receiving member 18 in the direction P (FIG. 1). The locking mechanism may then be manipulated (e.g., tightened) to lock or secure the second pin receiving member in a desired orientation and/or position. As noted above, the second pin receiving member 18 may be adjusted to precisely position and advance the pins 26, 28 into the first metacarpal before and during a procedure.

As shown in FIG. 2, the guide body 12 has first and second opposed faces (or primary surfaces) 12₁, 12₂. As illustrated, the first pin receiving member 14 is fixedly connected to the first face 12₁ and the second pin receiving member 18 is adjustably connected to the first face 12₁. The second pin receiving member projection 18p may extend through the slot 30 and past the second face 12₂, and the locking mechanism 32 may be configured to engage the second face 12₂ to lock or secure the second pin receiving member 18 in a desired orientation or position. Other configurations are contemplated. For example, the second pin receiving member 18 may be fixedly connected to the guide body 12 and the first pin receiving member 14 may be adjustably connected to the guide body 12. Alternatively, both the first and second pin receiving members 14, 18 may be adjustably connected to the guide body 12. Slides, rails and other movement control configurations may also be used.

Referring again to FIG. 3, and as described above, the first pin 22 can be precisely positioned and advanced into the trapezium T. The second pin 26 and then the third pin 28 may be precisely positioned and advanced into the first metacarpal MC using the pin guide 10. As noted above, these operations may be aided by fluoroscopy or the like. The pins 22, 28 may be used as guides for the drilling of channels or holes in the trapezium and first metacarpal, respectively. The guide 10 may be removed after the pins 22, 28 are correctly positioned and prior to drilling the holes.

Turning to FIG. 4, a trapezium channel or hole 40 drilled in the trapezium T and a first metacarpal channel or hole 42 drilled in the first metacarpal MC are illustrated. A graft 44 passes through the drilled holes 40, 42. The graft 44 can be anchored in the trapezium hole 40 by a biocomposite screw 46 or other anchor and anchored in the first metacarpal hole 42 by a biocomposite screw 48 or other anchor. In some embodiments, the biocomposite screw 46 is a 3 mm screw and the biocomposite screw 46 is a 4 mm screw. In some embodiments, the biocomposite screw 46 and/or the biocomposite screw 48 is a biotenodesis screw. In some embodiments, the graft 44 is a tendon graft and, in some embodiments, is a palmaris longis graft. In some embodiments, a length 50 of the graft 44 is anchored in the trapezium channel or hole 40 by the screw 46 and first 52 and second 54 opposite ends of the graft 44 are anchored in the first metacarpal channel or hole 42 by the screw 48.

### Exemplary Surgical Procedure

A short incision is made over the dorsal aspect of the trapezio-metacarpal joint of the thumb (FIG. 6). The joint is located with a syringe needle but the capsule/ligaments are not elevated and the joint is not entered. Retract the radial artery. Use the small straight periosteal elevator to clear the soft tissue off the radial aspect of the trapezium and palpate the trapezial ridge volarly. Under fluoro control, a 1.04 mm guide pin for the 3 mm bioabsorbable screw is inserted 5-6 mm proximal to the joint on the dorso-radial aspect of the trapezium and directed volarly toward the volar radial trapezial ridge. X-rays can be performed in the AP plane (palm up). The direction of the pin is parallel with the joint surface (syringe needle) and the radial wall of the trapezium. The desired volar exit is just radial to the FCR tendon through the trapezial ridge. The trapezial ridge is located on the volar-radial aspect of the trapezium. Use the small curved elevator to sweep the muscle off the volar capsule of the CMC joint. Once the pin penetrates the volar trapezium, you should be able to palpate the pin with a small curved periosteal elevator. Use the fluoro to locate pin placement. The trapezial pins should exit the volar cortex just proximal to the articular surface of the CMC joint. Once the pin is verified with fluoro, a guide (e.g., the guide 10 described above) is slipped over the trapezial pin and a second 1.04 mm pin is inserted in the dorsal center of the base of the 1st metacarpal 6-7 mm distal to the joint, directed toward the volar ulnar corner of the 1st metacarpal (FIG. 9). The smaller 1.04 mm pin inserted through the removable sleeve (e.g., the removable sleeve 24 described above). The pin in the base of the first metacarpal should exit volar, center ulnar, within the volar lip of the base of the first metacarpal. The two pins are parallel to slightly converging. If the position of the pin is satisfactory through fluoroscopy, the first MC pin and the sleeve are removed from the guide and are replaced by a 2.4 mm pin (FIG. 10A).

The guide is removed and a 3 mm hole is drilled in the trapezium (FIG. 10B) and a 4 mm hole is drilled in the 1st metacarpal (FIG. 10C). Use a small rasp and curette to widen the volar hole (distal edge) of the trapezium and volar hole (proximal edge) of the first metacarpal. A 90 degree curved suture/nitinol passer (FIG. 11) is inserted in the trapezial 3 mm hole (FIG. 12). Introduce the nitinol wire in the suture passer and feed ample out the other end. Use a grabber in the 1st metacarpal hole to secure the nitinol wire. (First grab the end of the suture passer, slightly release, slightly withdraw the passer and grab the wire.) Withdraw the nitinol passer. Attach fiber tape to the nitinol wire and pass through holes. Use the fiber tape to clear the muscle and soft tissue off the volar CMC joint capsule. Pass a suture (3-0 Fiberwire or Maxbraid) attached to a palmaris longus graft from proximal to distal. Place a clamp on the free end of the graft for safety. Pass the attached suture through the knot pusher end (FIG. 13). Use the knot pusher in the 1st metacarpal hole, pull the tendon through the trapezial hole, then the 1st MC hole. The tendon is secured in the trapezial hole with a 3 mm biocomposite screw leaving a minimum 2 cm free end of the proximal tendon graft (FIG. 17). Attach suture (3-0 Fiberwire or Maxbraid) at the appropriate location on the proximal tendon end for insertion with a 4 mm biocomposite screw and inserter handle. The joint is reduced and tension is applied to the distal tendon graft at the dorsal base of the 1st metacarpal while the 4 mm biocomposite screw is inserted, attached to the proximal tendon end (FIG. 18). This anchors two tendon ends in the first metacarpal and completes the circumferential AOL and DRL augmentation of the CMC joint. Usually the taut tendon graft is seen with redundant DRL/capsule next to it dorsally, indicating a tight augmentation. Cut off the excess tendon and suture to the trapezium. Close the skin and cast for a defined time (e.g., 3 weeks).

Embodiments of the invention include a pin guide and surgical procedure using the same for reconstruction of the AOL and DRL. The surgical procedure utilizes a tendon graft which is passed through drill holes in the trapezium and 1st MC. The tendon graft passes along the normal course of the AOL and DRL. The pin guide precisely positions the drill holes through which the tendon graft passes, so that the graft matches the anatomic location of the ligaments to be reconstructed. The pin guide is specifically calibrated to accept pins for the exact drills necessary to make the correct hole sizes for anchoring biotenodesis screws, for example. The trapezial pin can be 1.04 mm and the trapezial hole can be 3 mm, for example. The metacarpal pin can be 2.4 mm and the metacarpal hole can be 4 mm, for example. The guide is also adjustable for the variation in anatomy size for different patients. The guide can be made of many different biocompatible and/or medical grade materials including metal, plastic, rubber, ceramic, pyrocarbon and polyethylene.

Embodiments of the invention facilitate a minimally-invasive procedure which stabilizes the CMC joint, reduces pain, and potentially slows the progression of degenerative disease. The procedure requires a short period of immobilization and does not preclude performing a more definitive procedure at a later date.

Many alterations and modifications may be made by those having ordinary skill in the art, given the benefit of present disclosure, without departing from the spirit and scope of the invention. Therefore, it must be understood that the illustrated embodiments have been set forth only for the purposes of example, and that it should not be taken as limiting the invention as defined by the following claims. The following claims, therefore, are to be read to include not only the combination of elements which are literally set forth but all equivalent elements for performing substantially the same function in substantially the same way to obtain substantially the same result. The claims are thus to be understood to include what is specifically illustrated and described above, what is conceptually equivalent, and also what incorporates the essential idea of the invention.

1. A surgical pin guide for reconstruction of a carpo-metacarpal (CMC) joint of a hand, the pin guide comprising:
a guide body having first and second opposed second end portions;
a first pin receiving member connected to the first end portion of the guide body, the first pin receiving member having an elongated channel extending therethrough; and
a second pin receiving member connected to the second end portion of the guide body, the second pin receiving member having an elongated channel extending therethrough;
wherein the first pin receiving member channel is sized and configured to receive a proximal end portion of a first pin that has a distal end portion residing in a trapezium of a hand, and wherein the second pin receiving member channel is sized and configured to receive and align a second pin for insertion into a first metacarpal of the hand.

2. The pin guide of claim 1, further comprising a removable sleeve within the second pin receiving member channel, the removable sleeve having an elongated channel extending therethrough, wherein the removable sleeve channel is sized and configured to receive the second pin, and wherein, when the removable sleeve is removed, the second pin receiving member channel is sized configured to receive and align a third pin for insertion into the first metacarpal of the hand.

3. The pin guide of claim 2, wherein the third pin has a diameter that is greater than a diameter of the second pin.

4. The pin guide of claim 2, wherein:
the first pin receiving member channel has a diameter of between about 1 mm and 1.55 mm;
the removable sleeve member channel has a diameter of between about 1 mm and 1.55 mm; and
the second pin receiving member channel has a diameter of between about 2.2 mm and 2.55 mm.

5. The pin guide of claim 1, wherein the first pin receiving member is fixedly connected to the guide body.

6. The pin guide of claim 1, wherein the second pin receiving member is adjustably connected to the guide body.

7. The pin guide of claim 6, wherein the second pin receiving member is slidable and/or translatable along a length direction of the guide body away from and toward the first pin receiving member.

8. The pin guide of claim 7, wherein the guide body includes an elongated slot that receives a projection of the second pin receiving member, the projection being slidable within the slot.

9. The pin guide of claim 6, wherein the first pin receiving member channel defines a first axis, wherein the second pin receiving member channel defines a second axis, and wherein the second pin receiving member is pivotable so as to adjust an angle between the first axis and the second axis.

10. The pin guide of claim 6, further comprising a locking mechanism for locking and/or securing the second pin receiving member in place on the guide body.

11. The pin guide of claim 10, wherein the guide body has first and second opposing faces, the second pin receiving member residing adjacent the first face and the locking mechanism residing against or adjacent the second face.

12. The pin guide of claim 11, wherein the first pin receiving member is fixedly disposed on or integrally formed with the first face.

13. The pin guide of claim 1, wherein the guide body has an arcuate profile when viewed from the side.

14. The pin guide of claim 1, wherein the guide body, the first pin receiving member and/or the second pin receiving member is polymeric.

15. A method for performing anterior oblique ligament (AOL) and dorsoradial ligament (DRL) reconstruction of the carpo-metacarpal (CMC) joint of a patient, the method comprising:
advancing a distal end portion of a first guide pin in a trapezium of the patient;
positioning a guide having first and second pin receiving members such that a proximal end of the first guide pin is received in the first pin receiving member;
inserting a distal end portion of a second guide pin in the second pin receiving member of the guide; and
advancing the distal end portion of the second guide pin through the second pin receiving member of the guide and into a first metacarpal of the patient.

16. The method of claim 15, further comprising:
retracting the distal end portion of the second guide pin from the first metacarpal of the patient and from the second pin receiving member of the guide;
inserting a distal end portion of a third guide pin in the second pin receiving member of the guide; and
advancing the distal end portion of the third guide pin through the second pin receiving member of the guide and into the first metacarpal of the patient.

17. (canceled)

18. (canceled)

19. The method of claim 16, further comprising removing the guide after advancing the distal end portion of the third guide pin through the second pin receiving member of the guide and into the first metacarpal of the patient.

20. The method of claim 19, further comprising:
drilling a channel or hole in the trapezium using the first guide pin; and
drilling a channel or hole in the first metacarpal using the third guide pin.

21. The method of claim 20, further comprising removing the first and third guide pins after drilling the holes in the trapezium and the first metacarpal.

22. The method of claim 21, further comprising passing a graft through the hole drilled in the trapezium and the hole drilled in the first metacarpal.

23. (canceled)

24. The method of claim 22, further comprising:
anchoring the graft in the hole drilled in the trapezium using a first screw; and
anchoring the graft in the hole drilled in the first metacarpal using a second screw.

25. (canceled)

26. (canceled)

27. (canceled)

28. (canceled)
